(19) United States
(12) Patent Application Publication
(10) Pub. No.: US 2025/0011699 A1
(43) Pub. Date: Jan. 9, 2025

(54) DUAL-PROCESS SOLVENT AND SUGAR PRODUCTION PLANTS

(71) Applicant: Whitefox Technologies Limited, London (GB)

(72) Inventors: Virginia ANDRADE, Calgary (CA); Camilla Oliveira, Calgary (CA); Stephan BLUM, Calgary (CA); Thiago RIGHI, Calgary (CA)

(73) Assignee: Whitefox Technologies Limited, London (GB)

(21) Appl. No.: 18/761,570

(22) Filed: Jul. 2, 2024

Related U.S. Application Data
(60) Provisional application No. 63/524,786, filed on Jul. 3, 2023.

(51) Int. Cl. C12M 1/00; B01D 1/26; B01D 3/00; B01D 3/14; B01D 3/36; C07C 29/80; C12P 7/06; C13B 25/00

(52) U.S. Cl. CPC C12M 21/12; B01D 1/26; B01D 3/002; B01D 3/007; B01D 3/145; B01D 3/148; B01D 3/36; C07C 29/80; C12P 7/06; C13B 25/00

(57) ABSTRACT

Dual-process solvent and sugar production plants are provided in which inputs to the solvent production sub-system are received from the sugar product sub-system, which are both driven and supplied inputs from a shared pre-evaporator system. Improvements to the heat integration and dehydration technologies available for use in the plants are also provided, which may be used for initial construction, retrofit, replacement, or expansion of previous separation sections in the plants.

### CROSS-REFERENCE TO RELATED APPLICATION

The present application claims the benefit of and priority to U.S. Provisional Patent Application No. 63/524,786 titled “DUAL-PROCESS SOLVENT AND SUGAR PRODUCTION PLANTS” and filed on Jul. 3, 2023, which is incorporated herein in its entirety.

### BACKGROUND

To produce an organic solvent such as fuel grade ethanol, water and solids from fermentation must be removed. Typical processes use a series of distillation steps combined with a dehydration unit operation to achieve 99 vol. % or higher organic solvent content depending on specifications.

Ethanol produced from sugarcane must be concentrated to >99 wt % to be blended with gasoline and used in gasoline-driven engines. Given that this level purity cannot be achieved by conventional distillation due to an azeotrope forming between the ethanol/water mixture at ethanol concentration of around 95%, the most common dehydration technology used in sugarcane-based ethanol production is azeotropic distillation with cyclohexane.

The azeotropic distillation process is a type of distillation process used for separation of a component from a liquid mixture where an azeotrope forms (e.g., ethanol from an ethanol/water mixture). An azeotrope forms when the composition of vapors formed from the boiling of a mixture contains the same composition as the liquid at any given temperature and pressure. Thus, once an azeotrope forms, conventional distillation can no longer separate the components further. To perform the separation, azeotropic distillation is applied to break the azeotrope formed. The most common practice is the addition of an additional component known as an entrainer. When an entrainer is added to the mixture, the main azeotrope is broken and a new one is formed between the entrainer and any one or more components. For example, in the azeotropic distillation of an ethanol/water mixture, cyclohexane is typically used as the entrainer. In a first distillation column, known as azeotropic column, cyclohexane and hydrous ethanol are added. Cyclohexane forms a new tertiary heterogeneous azeotrope with ethanol and water. As a result of this new azeotrope having a lower boiling point than ethanol, anhydrous ethanol is removed from the column bottoms and a mixture of cyclohexane, water, and residual ethanol vapors is removed from the column top as an overhead stream, which is condensed and cooled before being forwarded to a decanter to undergo liquid-liquid separation. This heterogenous mixture separates in the decanter into an organic layer rich in the entrainer (e.g., cyclohexane) and aqueous layer (both containing Water and residual Ethanol). The cyclohexane is recycled back to the azeotropic column, whereas the aqueous layer is sent to a second distillation column known as a recovery column.

In the recovery column, water is separated from the residual cyclohexane and ethanol in the column bottoms. The residual cyclohexane and ethanol from the column top of the recovery column is condensed and cooled with a portion being recycled back to the azeotropic column for further separation. The remaining portion is directed back to the recovery column as reflux.

Despite the high energy consumption and the health risks associated with the use of solvents/entrainers in separating ethanol from water, cyclohexane dehydration is the most common technology used in sugarcane mills. Other alternatives include extractive distillation and molecular sieve dehydration.

Extractive Distillation involves a separating agent (e.g., a third component) such as monoethyleneglycol (MEG), which is added to a first distillation column to alter the relative volatility of the ethanol/water feed mixture by changing the intermolecular interactions between the components, resulting in separation of ethanol in the overheads of the column as distillate. Typically, the solvent added in the extractive distillation column has a higher boiling point than either component of the feed mixture. As a result, the solvent/water mixture is removed in the bottoms of the extractive distillation column before being fed to a second distillation column where the solvent is recovered in the column bottoms and re-circulated back to the Extractive Distillation column for reuse (and the water is removed via an overhead stream).

Molecular sieve dehydration involves the use of an adsorption processes using an adsorbent material in a porous solid form (known as molecular sieves) that selectively adsorb water molecules while a solvent remains a non-diffusing component due to differing molecular sizes of water and the solvent. There are many types of adsorbents, which include synthetic zeolites, microporous charcoals, active carbons, as well as natural adsorbents, including cornmeal, straw, and sawdust. Pressure swing adsorption (PSA) consists of the two main steps which are adsorption (molecular sieve bed in online mode) and desorption (molecular sieve bed in regeneration mode). Molecular sieve beds need to be regenerated in every cycle to be ready for the adsorption part of the cycle, which complicates the use of molecular sieves in continuous process systems.

### SUMMARY

The present disclosure provides new and innovative systems and methods for organic solvent (e.g., ethanol) production is dual-process production facilities that also produce sugar from a shared input with the solvent.

Additional features and advantages of the disclosed method and apparatus are described in, and will be apparent from, the following Detailed Description and the Figures. The features and advantages described herein are not all-inclusive and, in particular, many additional features and advantages will be apparent to one of ordinary skill in the art in view of the figures and description. Moreover, it should be noted that the language used in the specification has been principally selected for readability and instructional purposes, and not to limit the scope of the inventive subject matter.

### BRIEF DESCRIPTION OF THE DRAWINGS

FIG. 1 is a block diagram of an example dual-process production plant that produces an organic solvent and sugar from a shared input of sugar-bearing vegetable matter, according to embodiments of the present disclosure.

FIG. 2 is a block diagram of an example sugar production section, as may be part of a dual-process production system, according to embodiments of the present disclosure.

FIG. 3 is a block diagram of an example solvent production section, as may be part of a dual-process production plant, according to embodiments of the present disclosure.

FIGS. 4A-4E are plumbing diagrams for portions of distillation and dehydration units of a solvent production section, according to embodiments of the present disclosure.

FIG. 5 is a plumbing diagram for a dehydration unit using a membrane filter, showing various heat recovery features, according to embodiments of the present disclosure.

FIG. 6 is a flowchart of an example method for operating a dual-process production plant, according to embodiments of the present disclosure.

### DETAILED DESCRIPTION

The provided distillation and dehydration system is configured to produce an anhydrous organic solvent (e.g., >99% vol) and sugar from a shared sugar-bearing vegetable matter feedstock (e.g., sugarcane or sugar beets). In the present disclosure, the provided system is generally described in connection with the production of refined sugar and anhydrous ethanol primarily from sugarcane as the feedstock; however, several intermediate products of the system may also be collected for use as outputs (e.g., molasses at various grades, various unrefined or raw sugars at various grades,

The present disclosure contemplates that one of ordinary skill in the art will be familiar with the various grades of the products of the system referred to herein. For the avoidance of doubt, ethanol is referred to by various proof levels of 100 proof (100 P), 120 proof (120 P), 190 proof (190 P), and 200 proof (200 P)) that define purity levels for approximately at least 50% ethanol by volume, at least 60% ethanol by volume, approximately at least 95% ethanol by volume, and at least 99% ethanol by volume, respectively, but other purity levels may be specified for use according to the present disclosure. The present disclosure contemplates that different jurisdictions and technical field have standards that grade ethanol (and the additives or impurities therein) for different uses according to various scales that one of ordinary skill in the art will be familiar with. Generally, however, these standards define high-grade ethanol (e.g., for medical use) to be a higher purity/proof than fuel-grade ethanol (e.g., for use as a fuel or additive to another fuel).

For avoidance of doubt, definitions for the purity of sugar and the grade of molasses shall be consistent with those set by the International Commission for Uniform Methods of Sugar Analysis (ICUMSA) unless explicitly stated otherwise.

Additionally, various materials may be referred to herein as “freed” of another material (e.g., solids-freed, solvent-freed, water-freed), indicating that the first material has been distilled, filtered, or otherwise separated to remove (or be freed of) at least a portion of the second material. For example, a base liquid containing fifty percent water and fifty percent of an organic solvent (e.g., ethanol) may be subject to a first distillation process to produce a first water-freed stream of thirty percent water and seventy percent of the organic solvent, which may be subject to a second distillation process to produce a second water freed-stream of ten percent water and ninety percent of the organic solvent. In contrast, various materials may be referred to herein as “enriched” with another material (e.g., solvent enriched), indicating that the first material has been distilled, filtered, concentrated, or otherwise supplemented to increase a concentration of the second material. Using the previous examples, the water-freed streams may also be considered to be solvent enriched streams, and the remaining base material (from which the solvent enriched streams were separated) may be considered to be water enriched streams in comparison to their respective inputs.

As will be appreciated, a solvent production plant may operate in a continuous flow, or in discrete batches. Accordingly, various liquids or gasses may be transported as streams between the various elements of the plants, which may be continuously or discontinuously delivered to different elements via appropriate piping or ducting. Therefore, the present disclosure contemplates that the person of ordinary skill in the art will understand that various valves, splitters, collection vessels, and other flow-control elements may be used to selectively configure the pipes and ducts of the plant to deliver different amounts of inputs and outputs at different times. Accordingly, each of the illustrated examples of solvent production plants described herein may represent several different plants or a single plant at different times with elements that are inactive or selectively disconnected not illustrated to better describe and highlight the elements that are active and selectively connected. Stated differently, each example, unless stated explicitly otherwise or apparent from the context, represents a configuration or arrangement of a unified embodiment of the present disclosure.

When discussing the various streams, the present disclosure contemplates that all or a portion of a described stream can be delivered between a first element and a second element. Accordingly, as used herein when discussing that an identified stream is directed between a first element and a second element, the present disclosure contemplates that at least a portion of the identified stream is being directed as described.

For example, column A that produces stream A may direct stream A exclusively to column B at a first time and exclusively to column C at a second time, and column A may be understood as being configured to direct stream A to column B and column C (e.g., as time-based first/second portions) regardless of which column is currently receiving stream A. Similarly, column D that produces stream D may simultaneously direct stream D to column E and to column F, and may be understood as being configured to direct stream D to column E and to column F (e.g., as flow-based first/second portions) regardless of what portion of stream D is provided to column E or column F at a given time. Similarly, column G that produces and directs stream G only to column H, but may be throttled to provide any portion between 0-100% of a rated flow for stream G over a given time, may be understood as being configured to direct stream G to column H, regardless of what the flow rate is at any given time. Each of these examples shall be understood to describe a first column configured to direct a stream to a second column, regardless of whether the first column is also configured to direct that stream to a third column (at the same or different times) and regardless of what portion of the stream is so directed to any column.

Accordingly, a reference to the delivery, direction, receipt, or other action performed in relation to a stream shall be understood to refer to that action in relation to some or all of the identified stream. Additionally, discussion of an action performed in relation to a stream shall be understood as occurring at a given time, and that same action may be performed continuously across several times or discontinuously (e.g., not performed or performed differently at a second time). For example, a stream may be described as being directed to element A, while a second portion of that stream is directed to element B (either contemporaneously or at a different time), and that same stream may be directed to element A from time T₁ to time T₂, but not directed to element A from time T₂ to time T₃.

Various components of the presently disclosed systems may be in fluid communication with one another, such as through piping or ducting. The terms “piping,” “pipes,” “pipework,” “ducts,” “ducting,” “ductwork,” and “plumbing” are used interchangeably herein to refer to elements used to transport various fluids and solids throughout the described plant between or through other names elements, and may include (or omit) insulation to maintain the temperature (or allow the change of temperature) of fluids carried therein. One of ordinary skill in the relevant art will be able to select the materials and gauges of the piping or ducting based on the fluid flow requirements (e.g., flow rate, temperature, pressure, chemical composition) between two elements. Additionally, based on the positions of the various elements in the described plants and desired flowrates among the various points in the plants, one of ordinary skill in the relevant art is expected to be able to route the various pipes and fluids carried therein (e.g., via valve states) to achieve fluid communication among the various components as described herein. Accordingly, the piping may be arranged to selectively configure two or more components to be in communication with one another to direct, send, receive, etc. various inputs and outputs. Two components in fluid communication with one another may be in direct fluid communication (e.g., piping or ducting directly connects the two components without intermediate components other than valves or collectors) or may have intermediate components for processing a fluid between the two components, such as filters, pumps, heaters, odor removal vessels, coolers, condensers, vaporizers, etc.

The provided system includes vapor recompression (e.g., a mechanical or thermal vapor recompression unit) to recover heat from a rectification-distillation section (e.g., a rectifier/stripper column, wine column, extractive or azerotropic columns, recovery columns, or any other distillation column) or to upgrade low-pressure driving vapor or steam to a higher medium/high-pressure for used in other systems (e.g., distillation column feeding a dehydration unit, such as a membrane). The addition of vapor recompression enables further heat recovery within a stream by increasing the condensation temperature and pressure of that stream and later using its latent heat by condensing it. Vapor recompression units compress a vapor from a lower pressure to a higher pressure. Examples include compressors driven by engines, driven by electrical energy and motors (both referred to as MVR or mechanical vapor recompression units), or thermal vapor recompression units that forgo electrical power and instead use motive steam to entrain and compress the lower pressure vapor to a higher pressure (e.g., a steam jet pump).

Although the examples given herein may recite one type of compressor using one type of power source, different power sources or types of compressors can be used in a system design according to the operational constraints of the system. For example, different types of low-pressure stream or vapors can be upgraded to a higher pressure using compressors (e.g., one or both of Thermal Vapor Recompression (TVR) or Mechanical Vapor Recompression (MVR) compressors), and different types of high-pressure steams across the system can be used as motive fluids in addition to or alternatively to providing thermal energy. The energy savings provided by the addition of a vapor recompression unit in an organic solvent plant can be substantial when compared to the energy necessary to produce the steam that will be used in the plant.

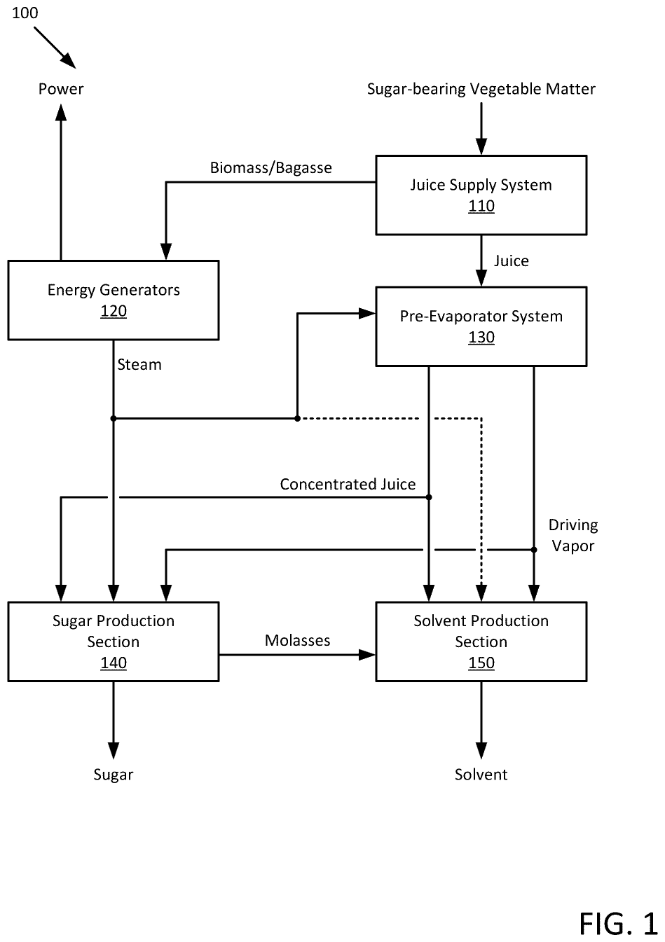

FIG. 1 is a block flow diagram of an example dual-process production plant 100 that produces an organic solvent and sugar from a shared input of sugar-bearing vegetable matter, according to embodiments of the present disclosure. In various embodiments, the organic solvent produced by the plant is 200 P ethanol, but various other concentrations/purities of ethanol or other alcohols or organic solvents Is (e.g., methanol, isopropanol, isobutanol) in various concentrations/purities may be produced in various embodiments or configuration of the described dual-process production plant 100.

To produce ethanol from vegetable matter, feedstock is crushed and turned into juice via a juice supply system 110. The feedstock may be various sugar-bearing vegetables such as sugarcane or sugar beets. The juice supply system 110 may slice, crush, masticate, or generally break the feedstock into smaller segments or pieces. Depending on the water content of the feedstock, the juice supply system 110 primarily from the juices extracted from the feedstock, but may also augment the liquid level of the juice with water or other fluids. Before being sent to fermentation, these feedstocks are passed through various refining processes to clarifying the juice and remove impurities. Unlike solvent production plants that use corn as a feedstock, which includes several process steps that occur at high temperatures (e.g., cooking and liquefaction to produce mash) and therefore require energy input before mash is sent to fermentation to produce beer, by using sugar-bearing vegetable feedstock, the juice is concentrated via the pre-evaporator system 130 before fermentation to produce wine.

The pre-evaporation process provides the driving vapor (also referred to as vegetal vapor) used to run the dual-process production plant 100. The pre-evaporator system 130 is driven by steam, and produces driving vapor that is used in multiple unit operations throughout the plant 100, such as evaporation and crystallization in sugar production and distillation and dehydration in ethanol production. After being concentrated, the juice goes to one or both of sugar production and solvent production. Unlike single-process plants, such as corn-based ethanol production plants, in which all of the mash is sent to fermentation for ethanol production, in a dual-process production plant 100, after being concentrated, the juice goes one or both of sugar production and solvent production.

In various embodiments, the juice supply system 110 includes various subsystems for clarification of the juice, in which impurities are removed. Accordingly, the juice supply system 110 can filter and separate the juice from the remnants of the feedstock, which is generally referred to as biomass or bagasse. The biomass/bagasse may be provided as a fuel input to one or more energy generators 120, such as biomass-fueled boilers connected to turbines to generate power and steam for use in the plant 100 or for delivery to a power grid or energy storage system (e.g., a flywheel, chemical battery, gravity-fed battery, etc.). In various embodiments, the biomass/bagasse may be supplemented with other fuels. Generally, biomass goes into boilers where the biomass is burned, and the heat from combustion is used to generate high pressure steam from steam condensate. The high pressure steam then goes to turbines in which a portion of the heat in the steam is converted into power by letting down the pressure (from high to medium and/or low pressure or vacuum). Medium/low pressure steam from the turbines is used in the plant for various purposes and to drive various reactions. This steam is called exhausted steam (vapor de escape).

For example, in various sugarcane-ethanol production plants, the main source of energy for running those plants comes from the combustion of bagasse or any available biomass. Because most of the processes in these sugarcane-ethanol plants are operated at pressures substantially equal to atmospheric pressure, electricity is produced from the high pressure steam generated in the bagasse boilers. This electricity can be used at the plant or be sold to the power grid. Low-pressure steam from the turbine is used to drive the pre-evaporator system 130. The driving vapor generated in the pre-evaporation system 130 can then be used to drive distillation and dehydration operations elsewhere in the plant. For example, this driving vapor is also used to drive the evaporators used in the sugar production section 140, and one or more distillation columns in the solvent production section 150. It is estimated that the distillation columns in a cyclohexane-based solvent production system consume approximately 3.3 kilograms (kg) of equivalent steam per liter (L) of anhydrous ethanol produced.

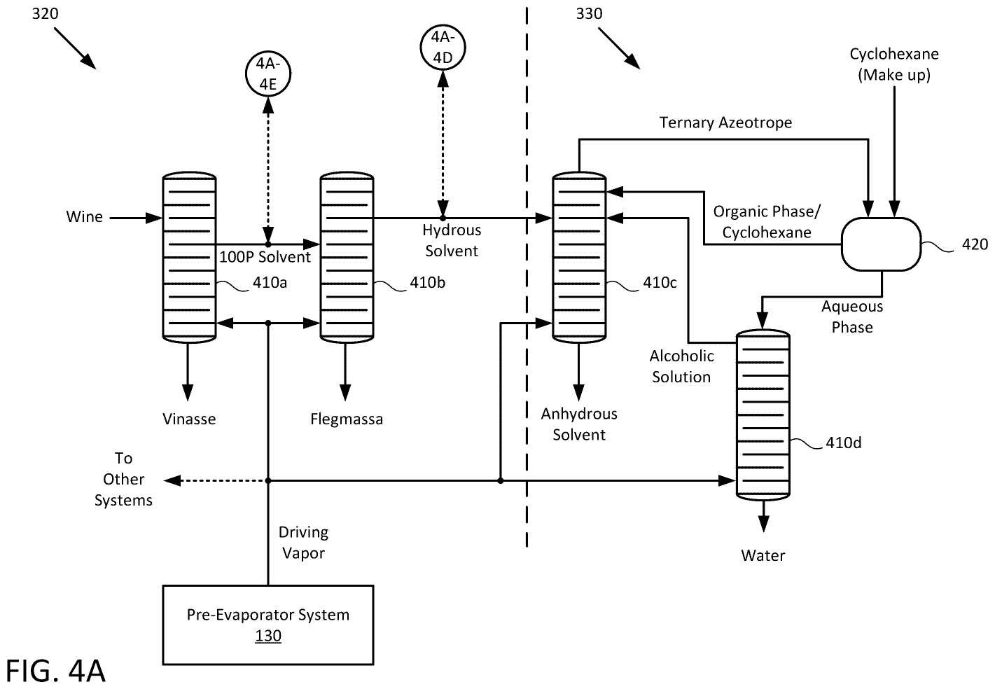

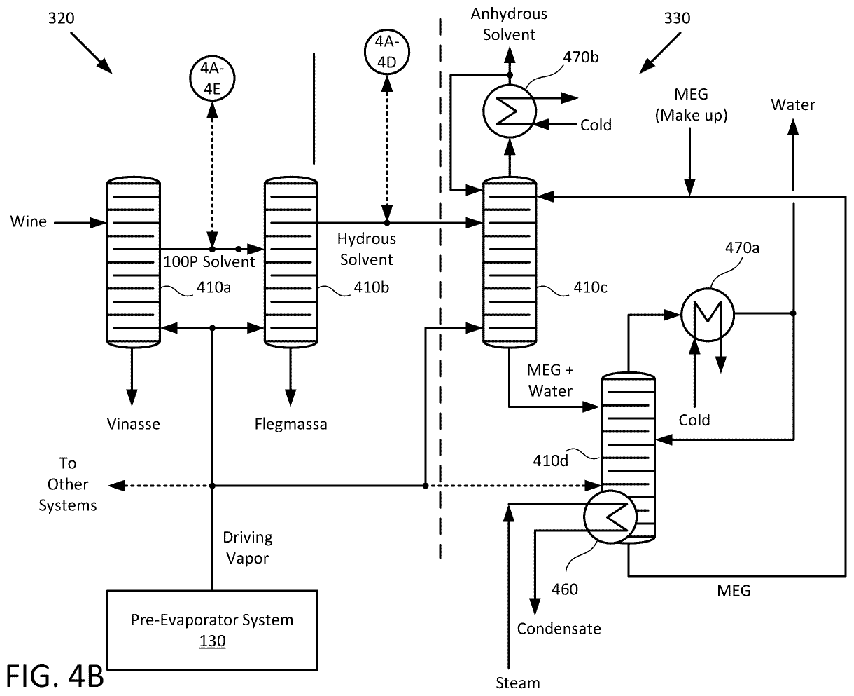

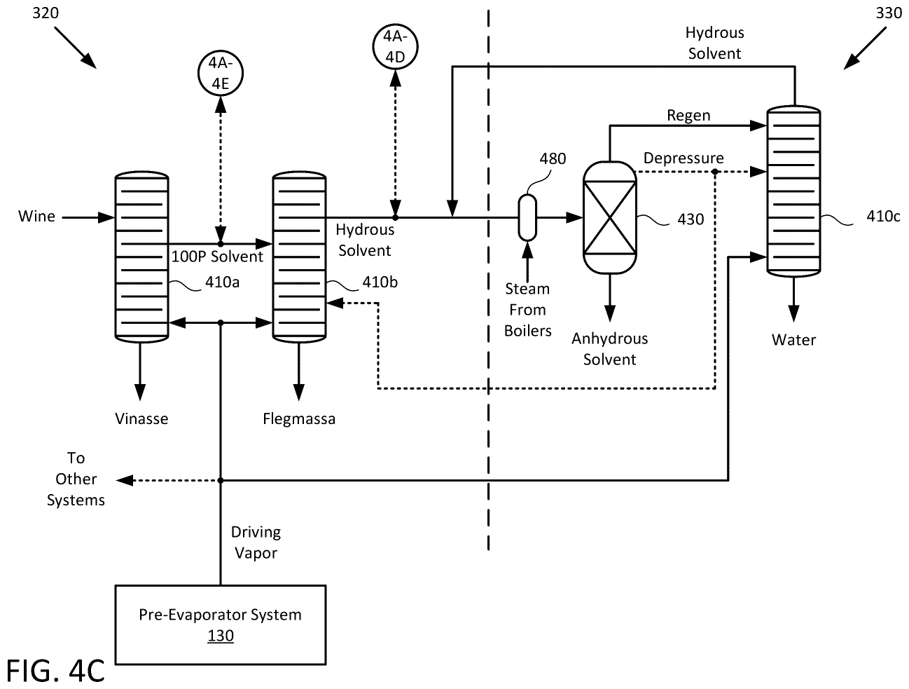

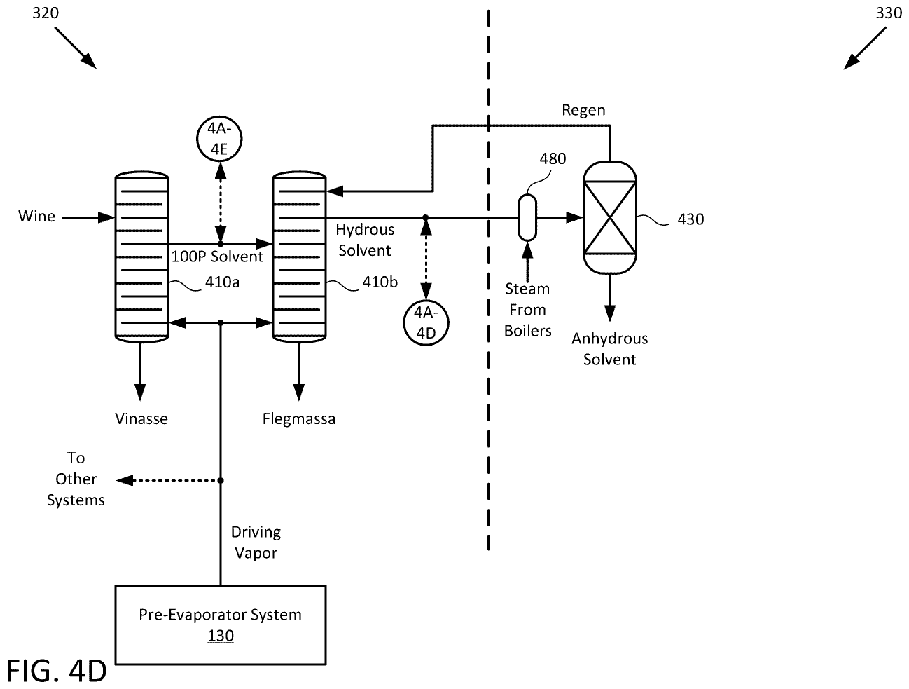

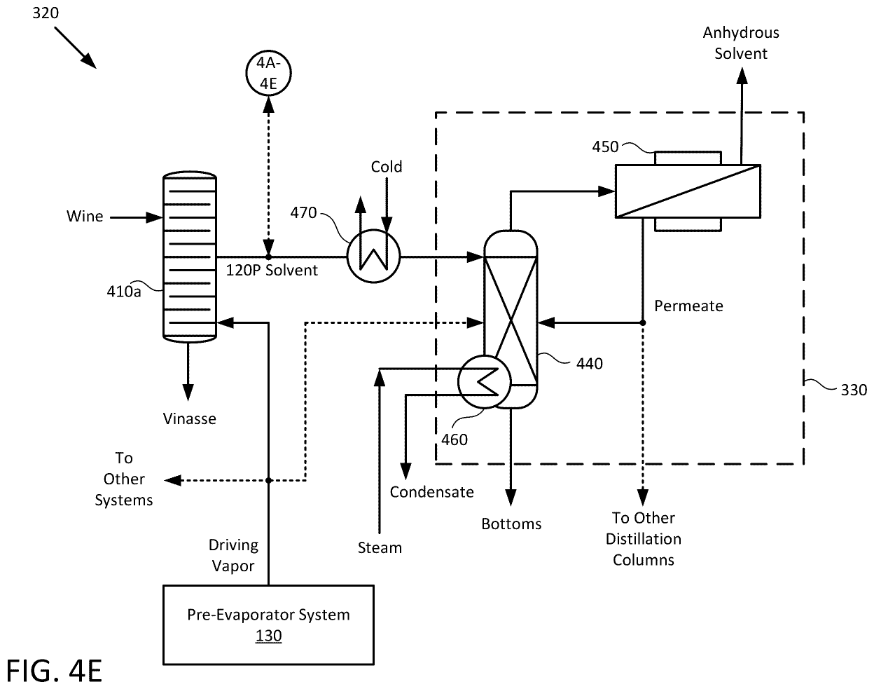

Generally, the high-pressure steam in a solvent production plant is generated in a boiler of the energy generators 120, which circulates back to the boiler as a condensate after transferring energy to the various other systems. The steam may be generated in the boiler at a first pressure (e.g., about 20 bar absolute (barA), which is used to spin a turbine to produce power, and is passed at a second pressure (e.g., about 2.5 barA) to other systems after the turbine (e.g., the pre-evaporator system 130, the sugar production section 140, the solvent production section 150). In some embodiments, different pressures of steams may be used in various downstream processes to drive distillation and drying processes in various columns. For example, a heat exchanger associated with a distillation column (as discussed in greater detail in regard to FIG. 4E) may receive steam at the same pressure as the turbine used to generate power. Also, a distillation column (as discussed in greater detail in regard to FIG. 4C or 4D) may receive steam from the boiler at a third pressure (e.g., about 10 barA) to separate water and a solvent. As will be appreciated, various compressors, expansion devices, and blow-off components may be included in the piping of the plant to assure safe operation at various desired pressures for the steam.

The driving vapor produced in the pre-evaporator system 130 is output at a lower pressure (e.g., about 1.7 barA) than the pressures of the steam. The driving vapor is provided to other systems downstream in the production process from the pre-evaporator system 130 (e.g., the sugar production section 140, the solvent production section 150) as a heat source or a motive force to drive distillation and drying processes in various columns. Using the lower-pressure driving vapor, which is close to atmospheric pressures (e.g., about 1 bar at 111 meters (m) altitude above sea level at 15 degrees Celsius (C)), and therefore is generally safer and can operate with thinner pipes/ducts than higher pressure steams or vapors. As will be appreciated, various compressors, expansion devices, and blow-off components may be included in the piping of the plant to assure safe operation at various desired pressures for the vapor.

A pre-evaporator system 130 includes one or more evaporators arranged in series (e.g., as a cascade), in parallel, or combinations thereof, and may be driven by the low-pressure steam produced by an energy generator 120 or steam plant (or high-pressure steam directly from a boiler if a turbine is omitted or bypassed). The pre-evaporator system 130 concentrates the juice received from the juice supply system 110 before directing the juice to the sugar production section 140, the solvent production section 150, or both. The pre-evaporator system 130 directs a portion of the juice with a higher concentration (e.g., a lower water content) to the sugar production section 140, while directing a less concentrated juice (e.g., a higher water content) to the solvent production section 150. In some embodiments, the pre-evaporator system 130 receives the juice with a dissolved sugar content of about 14 degrees Brix (Bx) and outputs the juice with a higher dissolved sugar content of about 25 degrees Bx.

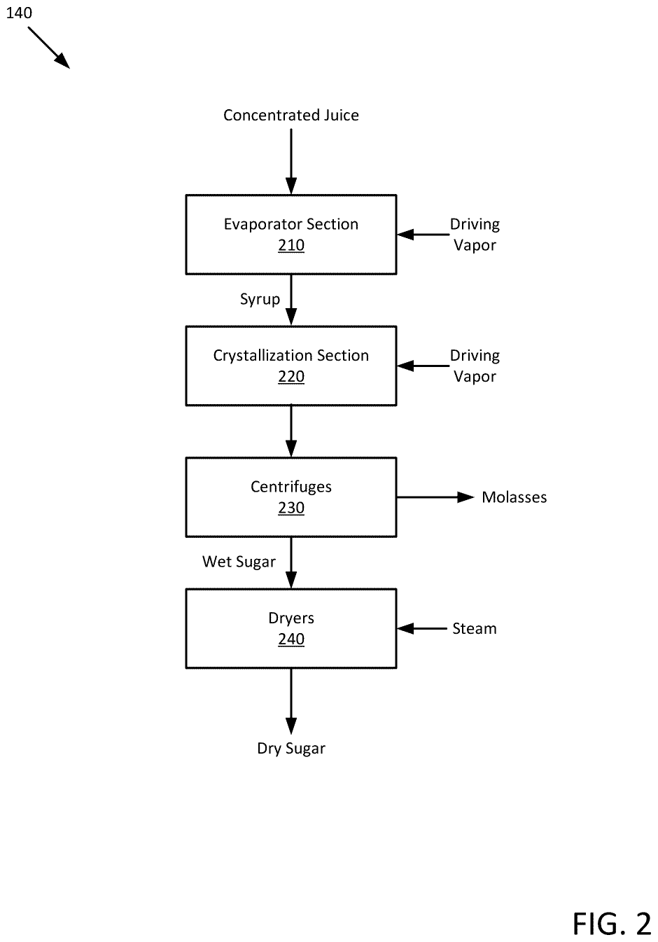

FIG. 2 is a block diagram of an example sugar production section 140, as may be part of a dual-process production plant 100, according to embodiments of the present disclosure.

The sugar production section 140 receives a concentrated juice sent from the pre-evaporator system 130 at an evaporation section 210 that includes a series of evaporators to produce a syrup of a higher concentration of sugar than the concentrated juice using the driving vapor.

The syrup continues to a crystallization section 220 and one or more centrifuges 230 to produce a wet sugar and molasses. The wet sugar continues to the dryers 240, which produce a dry sugar as an output. In various embodiments, the molasses may be provided as an output from the dual-process production plant 100, or may be used as an input to another process. For example, an operator may mix the molasses with the dry sugar to produce brown sugar of a desired grade. In another example, an operator may send molasses to a formation system of a solvent production plant to supplement the juice used in producing an organic solvent.

The present disclosure contemplates that various grades of sugars and molasses are known to persons of ordinary skill in the relevant art, and that the inclusion, omission, and operating conditions of the various elements and components of the sugar production section 140 to produce the desired grades of products will be understood.

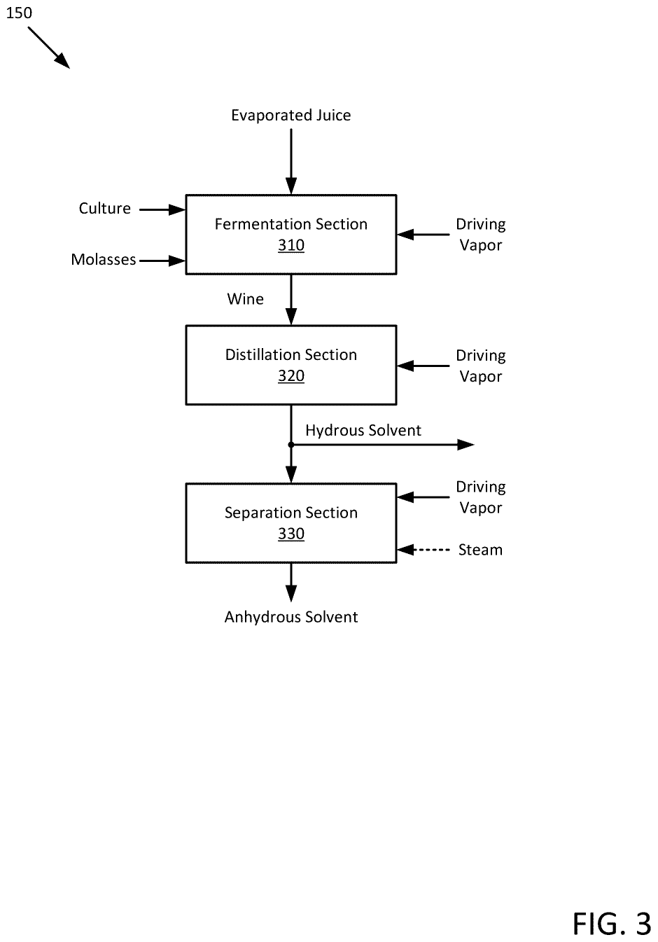

FIG. 3 is a block diagram of an example solvent production section 150, as may be part of a dual-process production plant 100, according to embodiments of the present disclosure.

The solvent production section 150 receives a concentrated juice sent from the pre-evaporator system 130 at a fermentation section 310 that includes one or more fermentation vessels (e.g., vessels in which fermentation takes place). In various embodiments, the fermentation vessels receive, or maintain, a culture of a yeast or bacteria that convert the sugars in the juice into the desired solvent (e.g., an alcohol). In some embodiments, the fermentation vessels receive molasses (which may be diluted or augmented with makeup fluid) from the sugar production section 140 to provide additional sugars and other feed for the sample to convert into the desired solvent. When converting sugars to a desired solvent of ethanol, the resulting fermentation product is referred to as a wine, which contains ethanol, water, and solids, and is sent to a distillation section 320 and a solvent/alcohol separation section 330 to separate the ethanol (or other desired solvent) from the water and solids.

The operation of the dual-process production plant 100 using wine may be contrasted to beer production in a corn-based ethanol production plant. Similarly to wine production, beer is generated from fermentation and the beer is sent to a distillation column (often referred to as a “beer column” or a “wine column” according to the fluid input). In each of the processes, the main objective is the separation of a portion of the water and solids from fermentation in the bottoms of the columns from the desired ethanol (or other solvent) in the beer or wine. However, the two columns operate on different solvent input streams due in part to the different feedstock, which may results in different concentrations of the solvent as input to the beer/wine columns, different off-products or concentrations thereof (e.g., different esters, undesired alcohols, spent yeast, etc.) that need to be removed via distillation from the beer and the wine respectively, different output concentrations of the solvent (e.g., due to the off-products), and combinations thereof. Energy for both beer/wine columns is provided by process vapors from evaporation; however, the beer column uses process vapor from evaporation that is part of distiller dray grain soluble (DDGS) feed production (which may be used as animal feed), while the wine column uses process vapor originated in a pre-evaporator system 130, which is absent from single-process plants, such as corn-based ethanol production plants. Additionally, in some cases, the beer column operates at vacuum pressures (e.g., about or below 0.7 barA), whereas the wine column operates at low pressures greater than atmospheric pressure (e.g., about 1-2 barA).

A byproduct of corn ethanol production is DDGS, while for sugarcane ethanol production, the outputs/byproducts include sugar, vinasse, and bagasse, which in a dual-process plant are used as products of the plant (e.g., as a commodity (sugar), fertilization/irrigation (vinasse), energy production (bagasse). For both cases, evaporation is used to concentrate thin stillage into DDGS and juice into sugar. In corn-based production, solids coming from the beer column bottoms are concentrated in evaporators that are mainly steam driven. The beer column is part of the ethanol production distillation train. In the sugarcane case, juice after pre-evaporation, which happens before the flows are split into the ethanol production and the sugar production trains, is further concentrated in a set of evaporators that are vapor driven and thereafter crystallization and drying is done to obtain sugar.

The processes in the distillation sections 320 yield a hydrous solvent (e.g., 190 P ethanol). To produce an anhydrous solvent (e.g., 200 P ethanol), the hydrous solvent is directed to a solvent separation section 330 to remove additional water. For example, in corn-based ethanol production, molecular sieves are often used as the main dehydration technology. In another example, in sugarcane-based ethanol production, cyclohexane is often used as the main dehydration technology; however, other types of dehydration such as monocthyleneglycol (MEG) injection, molecular sieves, or membrane filters can also be used for sugarcane-based ethanol production. Various layouts of distillation sections 320 and solvent separation sections 330 using different technologies are discussed in greater detail in regard to FIGS. 4A-4E.

FIGS. 4A-4E are plumbing diagrams for portions of distillation sections 320 and solvent separation section 330 of a solvent production section 150, according to embodiments of the present disclosure. In each of FIGS. 4A-4E, various distillation columns 410a-d (generally or collectively distillation column 410) are shown and the operation thereof is discussed. However, the present disclosure contemplates that each plumbing diagram may describe one or more systems that use a shared one or more wine columns 410a (also referred to as column A 410a) and a shared pre-evaporator system 130. Accordingly, each of the plumbing diagrams may be understood as separate layouts for the use of different technologies in the production of a solvent in different plants, as alternatives or additions to one another (e.g., as retrofits of additional technologies with existing technologies, as parallel processes to expand existing production capacity using installed technologies of a different type, etc.) in a single plant, or combinations thereof. Stated differently, the distillation sections 320 and the solvent separation sections 330 of a dual-process production plant 100 may include one or more of the distillation sections 320 and solvent separation sections 330 for which plumbing diagrams are shown in FIGS. 4A-4E.

In various embodiments, the distillation section 320 includes one or more distillation columns 410. Generally, a first distillation column (referred to as column A 410a) removes water and solids via a bottoms stream (referred to as vinasse) to generate a solvent-enriched vaporous overhead stream containing the solvent and water with a lower concentration of water and solids than the wine supplied to column A 410a from the pre-evaporators. For example, column A 410a may yield a water-freed overhead stream of 100 P-120 P (e.g., 100 P, 100 P-105 P, 105 P-110 P, 110 P-115 P, 115 P-120 P, or 120 P) ethanol. Column A is generally operated at or near atmospheric pressure (e.g., about 1 barA), and is driven by driving vapor generated in the pre-evaporator system 130.

In some embodiments, the vaporous overhead stream from Column A is sent to a second distillation column (referred to as Column B 410b) in which the solvent is further concentrated up to the azeotropic point. For example, hydrous ethanol (e.g., between 90-96% concentration; 180 P-192 P) is removed from the top of Column B 410b as a hydrous solvent overhead stream. The hydrous solvent overhead stream may be supplied as a product, be further dehydrated to produce anhydrous ethanol via the solvent separation section 330, or sent to additional distillation columns to remove specific impurities (or may be received from the additional distillation columns 410 that remove these impurities that are located between column A 410a and column B 410b). The bottom stream of column B 410b are mainly water (e.g., Flegmassa), and may be used as process water for the plant. The vaporous hydrous solvent stream output from Column B 410b is condensed and sent to a solvent separation section 330, which may include a distillation column 410 and one or more of cyclohexane processors, monoethyleneglycol (MEG) processors, molecular sieve units (MSU), or membrane filters as dehydration units, as discussed in greater detail in relation to FIGS. 4A-4E, respectively.

FIG. 4A illustrates a plumbing diagram for a distillation section 320 using a first distillation column (referred to as column A 410a) and a second distillation column (referred to as column B 410b) to produce a hydrous solvent, and a solvent separation section 330 including a cyclohexane-based dehydration unit to produce an anhydrous solvent. As illustrated, the distillation section 320 includes at least column A 410a and column B 410b to produce the hydrous solvent stream that is provided to the solvent separation section 330. Bottoms streams for the byproduct outputs from column A 410a and column B 410b include vinasse and flegmassa, respectively.

By way of example, in a corn-based ethanol plant, the output from a beer column is further concentrated in one or more rectification columns that are driven by process vapors and/or steam, while in a sugarcane-based ethanol production plant, column B 410b is driven by process vapors from the pre-evaporator system 130. Accordingly, the rectifier column for corn-based ethanol production operate at vacuum pressure (e.g., around 0.3-0.6 barA), while column B 410b in sugarcane-based ethanol production operates at approximately atmospheric pressure (e.g., 1 barA).

When using a cyclohexane-based dehydration unit, as in FIG. 4A, the hydrous solvent stream is directed to a third distillation column (referred to as column C 410c), where a cyclohexane or other entrainer stream is introduced to the hydrous solvent stream. Column C 410c outputs the dehydrated solvent as an anhydrous solvent stream (e.g., 200 P ethanol) as a bottoms stream, and a ternary azeotrope stream (e.g., an entrained water stream) as an overhead stream. A cyclohexane decanter 420 (e.g., a decanting tank) and a recovery distillation column (referred to as column D 410d) receive the entrained water stream and makeup (fresh) cyclohexane to release the cyclohexane from the entrained water (e.g., in the presence of H₂SO₄ as a catalyst) for reuse in column C 410c. In various embodiments, the cyclohexane decanter 420 returns an organic phase/cyclohexane stream to column C 410c, and directs an aqueous phase stream to column D 410d for further processing. Column D 410d removes the captured water as a bottoms stream, and returns an alcoholic solution (as an overhead stream) to column C 410c. Column C 410c and column D 410d may be driven via the driving vapor from the pre-evaporator system 130 by direct injection or thought a reboiler.

FIG. 4B illustrates a plumbing diagram for a distillation section 320 using a first distillation column (referred to as column A 410a) and a second distillation column (referred to as column B 410b) to produce a hydrous solvent, and a solvent separation section 330 including a MEG-based dehydration unit to produce an anhydrous solvent. As illustrated, the distillation section 320 includes at least column A 410a and column B 410b to produce the hydrous solvent stream that is provided to the solvent separation section 330. Bottoms streams for the byproduct outputs from column A 410a and column B 410b include vinasse and flegmassa, respectively.

When using a MEG-based dehydration unit, as in FIG. 4B, the hydrous solvent stream is directed to a third distillation column (referred to as column C 410c), where MEG is introduced as a separating agent. Column C 410c outputs the dehydrated solvent as an anhydrous solvent stream (e.g., 200 P ethanol) as an overhead stream, and an entrained stream of MEG and water as a bottoms stream. In various embodiments, a first condenser 470a (generally or collectively, condenser 470) is disposed in the piping between the overhead output of column C 410c and another system (e.g., a storage tank) to condense the vaporous anhydrous solvent into a liquid, or lower the temperature of the vaporous anhydrous solvent (e.g., against a cold stream to pre-heat for use elsewhere in the plant, as a driving vapor imparting thermal energy to another column, etc.). In various embodiments, a portion of the condensed anhydrous solvent that has passed through the first condenser 470a is returned to column C 410c as reflux. Column C 410c may be driven via the driving vapor from the pre-evaporator system 130, via direct injection or through a reboiler.

A recovery distillation column (referred to as column D 410d) receives the entrained water stream from column C 410c to release the MEG from the entrained water for reuse in column C (as a bottoms stream from column D), and to remove the captured water (as an overhead stream from column D 410d). In various embodiments, the water is removed from column D as a vaporous overhead stream, and may be condensed via a second condenser 470b into a liquid to be reintroduced to column D as reflux or sent to another part of the plant. In various embodiments, the vaporous water overhead stream may be a steam that is used as a hot source in the second condenser 470b to raise the temperature of another stream for use elsewhere in the plant, as a driving vapor imparting thermal energy to another column, or as a motive fluid. Column D 410d may be driven via the driving vapor from the pre-evaporator system 130.

FIG. 4C illustrates a plumbing diagram for a distillation section 320 using a first distillation column (referred to as column A 410a) and a second distillation column (referred to as column B 410b) to produce a hydrous solvent, and a solvent separation section 330 including an MSU-based dehydration unit to produce an anhydrous solvent. As illustrated, the distillation section 320 includes at least column A 410a and column B 410b to produce the hydrous solvent stream that is provided to the solvent separation section 330. Bottoms streams for the byproduct outputs from column A 410a and column B 410b include vinasse and flegmassa, respectively.

When using a MSU-based dehydration unit, as in FIG. 4C, the hydrous solvent stream is directed to an MSU 430, which may include one or more molecular sieve beds that are configured to generate a product streams of an anhydrous solvent and one or more regenerate streams from the hydrous solvent stream. In some embodiments, the one or more regenerate streams include a regen stream (also referred to as MSU-Regen) and a depressure stream. In various embodiments, a vaporizer 480 is disposed in the piping between column B 410b and the MSU 430 to increase the temperature or pressure of the hydrous solvent stream (or affect a phase change from liquid to vapor) before delivery to the MSU 430.

In various embodiments, the molecular sieve beds are filled with zeolite pellets, which adsorb water to produce anhydrous vapor until the zeolite pellets are saturated with water. A saturated zeolite pellet bed may be regenerated by directing a stream of freshly dehydrated organic solvent to contact a saturated zeolite pellet bed to thereby remove water from the saturated zeolite pellet bed by applying vacuum, which produces one or two regenerate stream; the regen stream and (optionally) the depressure stream. In instances in which the MSU 128 includes multiple zeolite pellet beds, a saturated zeolite pellet bed may be regenerated while an unsaturated zeolite pellet bed is used to contemporancously dehydrate a hydrous solvent stream. In at least some aspects, the product stream of the MSU 430 may be condensed (e.g., via one or more evaporators or condensers) and directed to a tank for storage.

The MSU regen stream may have an organic solvent concentration between 40-80 vol % of the solvent and therefore is recycled to upstream distillation for reprocessing. For example, the regen stream (and optionally the depressure stream) may be directed to column C 410c (e.g., a stripper column or a rectifier column). Column C 410c may output, as a bottoms stream, the water removed from the regen stream, and, as an overhead stream, a reprocessed hydrous solvent stream, which may be directed back as an input to the MSU 430, or used elsewhere in the production plant 100. The depressure stream may have a concentration above 80 vol % of organic solvent and (in some embodiments) may also be recycled to upstream distillation for reprocessing, such as to column B 410b, the MSU 430, or elsewhere in the production plant 100.

FIG. 4D illustrates a plumbing diagram for a distillation section 320 using a first distillation column (referred to as column A 410a) and a second distillation column (referred to as column B 410b) to produce a hydrous solvent, and a solvent separation section 330 including an MSU-based dehydration unit to produce an anhydrous solvent. As illustrated, the distillation section 320 includes at least column A 410a and column B 410b to produce the hydrous solvent stream that is provided to the solvent separation section 330. Bottoms streams for the byproduct outputs from column A 410a and column B 410b include vinasse and flegmassa, respectively.

The plumbing diagram of FIG. 4D uses an MSU-based dehydration unit, similarly to FIG. 4C, but the piping has been selectively configured to avoid using column C 410c, or the plant otherwise omits column C 410c from the design. Instead of routing the regen stream for further processing in column C, the MSU 430 routes the regen stream back to column B 410b for further processing.

FIG. 4E illustrates a plumbing diagram for a distillation section 320 using a first distillation column (referred to as column A 410a) and omitting or bypassing a second distillation column (referred to as column B 410b or as a rectification column) to produce a hydrous solvent, and a solvent separation section 330 including a membrane filter-based dehydration unit to produce an anhydrous solvent. In various embodiments, when bypassing column B 410b, column B may be repurposed as a second column A 410a (e.g., allowing a rectifier column to be retrofitted for use as a second wine column to increase plant throughput) or may be used for continued operation with a different dehydration technology (e.g., as discussed in relation to FIGS. 4A-4D), such as when expanding capacity for the plant by adding a membrane-based dehydration unit. Other options for re-using an otherwise bypassed column B 410b can include reconfiguring or cascading energy across the plant to obtain additional energy savings.

Although described as a “filter,” the membrane filter 450 does not operate by separating components based on the sizes thereof (e.g., via holes in a material sized to permit passage of a subset of the components), but rather by the chemical affinity of the components to an active layer of the material of the membrane filter 450.

By removing or bypassing the rectification column (e.g., Column B 410b), the dual-process production plant 100 can operate with less driving vapor (having fewer active distillation columns 410 to drive), and thus operates with reduced demand for bagasse as a fuel in the boilers. Although the design of the membrane filter-based dehydration unit decreases demand for steam and vapor from the energy generators 120 system, in some embodiments, the same amount of bagasse is produced from the same amount of feedstock to be produced, which allows for excess or surplus bagasse to be put to different uses. For example, bagasse may be output as a product of the plant (e.g., on the spot market), be used to produce surplus electricity (e.g., that is not required by a sugar or solvent production process) to be output to a power grid or used for other system in the plant (e.g., air conditioning, lighting, computing needs). Additionally or alternatively, the juice supply system 110 can attempt to extract more sugar from the feedstock before transferring the leftovers to the energy generators 120 as bagasse; extracting more sugar and less bagasse for the same volume of feedstock input. By reducing the amount of bagasse burnt, the amount of heat input required in burning it to generate heat and power for a sugar mill and ethanol production facility is also reduced, especially since bagasse contains a high moisture content (40-50% water by weight), which reduces the potential energy output from the bagasse as a fuel source. Additionally or alternatively, the “excess” bagasse can be used to produce more of the solvent when plant capacity allows for a higher throughput.

As will be noted, by omitting or bypassing column B 410b, the distillation section 320 transfers the solvent at a lower concentration to the separation section 330 than in configurations that include column B (e.g., 90 P-120 P versus 190 P for ethanol). For example, when using a membrane filter-based dehydration unit, as in FIG. 4E, the hydrous solvent stream (e.g., of 90 P, 90 P to 100 P, 100 P, 100 P to 120 P, or 120 P) is directed to a stripper distillation column (referred to as column S 440), where steam is directed through a heat exchanger 460 to generatheat the hydrous solvent stream to generate a vaporous overhead stream and drive separation of a bottoms stream from an overhead stream. In various embodiments, the overhead stream from column A 410a is directed through a condenser 470 before delivery to column S 440, to be received as a liquid rather than as a vapor (e.g., ˜50 v % to ˜90 v %).

Column S 440 generates an overhead vapor stream from an organic solvent-water concentrated feed stream that is then directed to contact the membrane filter 450 at a higher concentration of the solvent than is received by column S 440. For example, column S may receive 100 P ethanol and provide 160 P ethanol to the membrane filter 450. Column S 440 also generates a bottom stream mostly consisting of water that may be directed to another area of the plant. In various aspects, the bottom stream from column S 440 may be used to heat a suitable cold stream (e.g., steam condensate, process water, scrubber water, 190 P, a regenerate stream, a wine feed stream, etc.).

The membrane filter 450 continuously removes water from the overhead stream from column S 440 to produce a vaporous water-rich stream (also referred to as a permeate stream) and a vaporous anhydrous organic solvent-rich stream (also referred to as a retentate stream) of the anhydrous solvent. For example, the vaporous anhydrous ethanol-rich stream may include 99% by volume or higher of organic solvent. In some aspects, the membrane filter 450 may be a polymer membrane, which may be constructed from a plurality of hollow fibers. A selective layer may be placed on either the outside (e.g., shell side) or the inside (e.g., lumen side) of the hollow fibers. In other examples, the membrane filter 450 may have other suitable forms that suitably dehydrate a feed vapor stream as part of a high-grade organic solvent production process, such as tubular membranes including zeolites membranes or spiral wound membranes.

One of the features of the membrane filter 450 that contributes to the energy efficiency of the design is that dehydration can occur in a single pass in the membrane filter 450 from a higher water content feed (e.g., azeotrope agnostic operation) than in other dehydration technologies. Accordingly, there is no need to use a rectification process (which is an energy intensive operation) prior to dehydration. Therefore, column S 440 (positioned in the flowpath before the membrane filter 450) is mainly used to vaporize the feed to the membrane filter 450. In some embodiments, column S 440 may be omitted or bypassed, and a vaporizer is used to provide a vaporous feed to the membrane filter 450. With column S 440 omitted or bypassed, piping instead directs the permeate stream from the membrane filter 450 to another distillation column 410 in the plant 100 (e.g., column A 410a or column B 410b (if operational)) for reprocessing. Additionally, the ability to process feed streams with higher water content can allow a condenser 470 or compressor (e.g., using by TVR, MVR, or a combination thereof) to compress the overheads stream from column A 410a (other distillation column 410, or combination thereof). The resulting pressurized vapor can be fed directly to the membranes of the membrane filter 450 for dehydration in parallel with the overheads stream provided by columns S 440.

In at least some aspects, retentate vapor generated by the membrane filter 450 in the solvent separation section 330 system may be condensed for storage or use elsewhere as a liquid in the plant. For instance, the retentate vapor may be condensed via a condenser against any cold stream available, thereby condensing the retentate vapor into a liquid and heating another vapor or liquid to reducing steam or vapor demands for heating in the various processes across the plant. In some instances, the retentate vapor may be condensed via one or more of the evaporators (e.g., in the pre-evaporation system 130 or in the evaporators used for sugar production), or against a stream condensate from a condenser or heat exchanger to generate a low-pressure stream for use elsewhere in the plant 100. The condensed retentate liquid may be directed to a tank (e.g., a 200 P tank) for storage. In some aspects, prior to the condensed retentate liquid reaching the storage tank, the condensed retentate may be directed to a flash vessel where the produced 200-proof flash vapor stream can recover heat against another cold stream to be heated and be directed to a carbon dioxide (CO₂) removal system. The CO₂ removal system is a low-pressure flash vessel in which vapor and a liquid stream are generated. Retentate liquid energy may be further recovered against other process streams (e.g., permeate liquid, scrubber bottoms, liquid feed to the solvent separation system 330).

In various examples, the permeate vapor generated by the membrane filter 450 in the solvent separation section 330 may be condensed. For instance, the heat available in the permeate vapor may be used to heat a suitable cold stream (e.g., steam condensate, process water, scrubber water, 190 P, a regenerate stream, a wine feed stream, etc.) at a condenser, thereby condensing the permeate vapor into a liquid and heating another vapor or liquid to reducing steam or vapor demands for heating in the various processes across the plant.

In some examples, such as the one illustrated in FIG. 4E, the condensed permeate liquid may be directed back to the column S 440. The permeate liquid may be heated by a suitable hot stream (e.g., flash vapors, distillation column bottoms stream, retentate liquid, etc.) at a heat exchanger prior to being introduced into column S 440 in some embodiments. For instance, the permeate liquid may be heated by the retentate liquid at a heat exchanger.

Although, the permeate steam is described in FIG. 4E as being condensed and directed back to column S 440, the permeate stream may additionally or alternatively be directed to column A 410a, or a column B 410b or column C 410c in another one of FIGS. 4A-4C.

The use of membrane dehydration with vapor recompression can have advantages over the use of MSUs or other dehydration technology for dehydration of solvents. For instance, the use of membrane filters 450 for dehydration can provide an organic solvent plant with: (i) a stable and continuous distillation section by elimination of the molecular sieves regenerate streams, (ii) continuous operation compared to cyclic “batch” operation for regeneration of MSUs, (iii) a steady column overheads streams flow that can then be used by a vapor recompression unit to allow for heat recovery, (iv) a lower energy consumption in distillation by removing the treatment of additional streams from MSUs such as regen, and (v) a system that is modular and, therefore, easy to increase capacity by introducing additional cartridges of membranes filters 450.

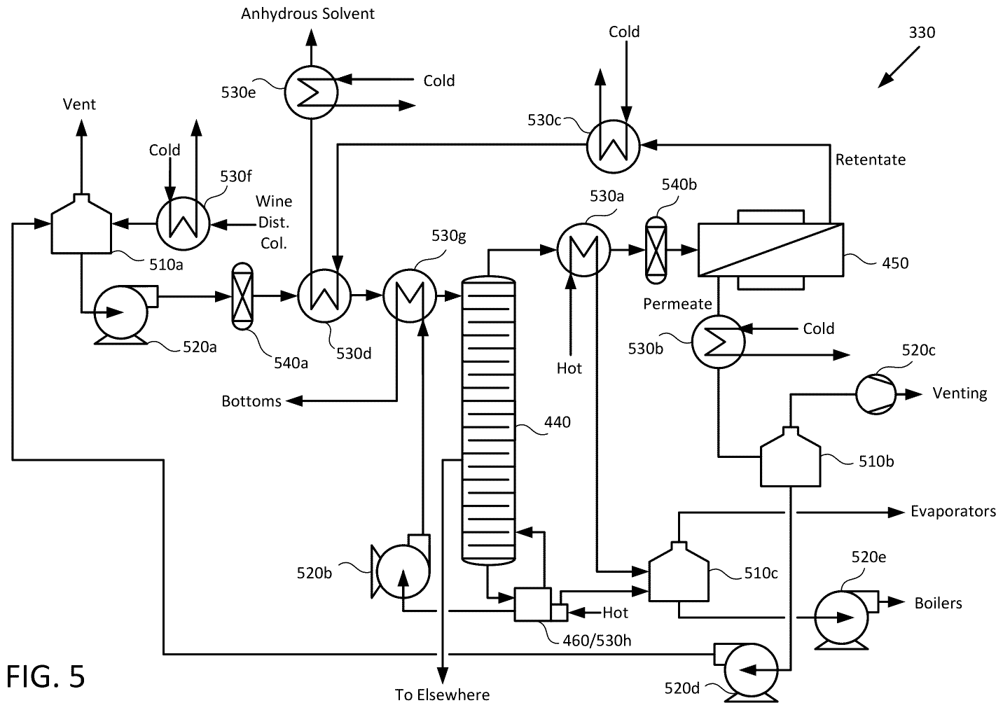

FIG. 5 is a plumbing diagram for a solvent separation section 330 using a membrane filter, showing various heat recovery features, according to embodiments of the present disclosure.

Dehydration by azeotropic distillation using an entrainer (e.g., cyclohexane as in FIG. 4A) can be eradicated completely by using the filter-based dehydration unit shown in FIG. 4E, which mitigates or eliminates the significant health risks associated with exposure to cyclohexane. Additionally, the use of the filter-based dehydration unit can provide various heat recovery options among the various fluids in the dual-process production plant 100, from (e.g., from available heat in the retentate, permeate or stripping column bottom streams for us in upstream processes). Thus, improving overall plant energy efficiency (reducing driving vapor and hence bagasse demand) and reducing operational expenditures.

The overheads stream received from column A 410a (e.g., at 50-60 wt % solvent concentration) by column S 440 may undergo several pre-cooling and pre-heating processes using the thermal energy in various intermediate or final product streams of the plant to reduce the energy expenditure to bring the wine to an appropriate temperature and pressure to generate the overhead stream for provision to the membrane filter 450. As shown, column A overheads received from column A 410a may be condensed/cooled for storage in a first tank 510a (generally or collectively, tank 510) against a cold stream in a sixth heat exchanger 530f (generally or collectively, heat exchanger 530). As used herein, a cold stream refers to any fluid used to cool another fluid of a higher initial temperature in a heat exchanger 530. Similarly, as used herein, a hot stream refers to any fluid used to heat another fluid of a lower initial temperature in a heat exchanger 530. As will be appreciated, each heat exchanger 530 receives at least one cold stream and at least one hot stream to equalize the temperatures of the streams from input to output without mixing the two fluids.

A first pump 520a (generally or collectively, pump 520) draws the stored solvent from the first tank 510a to feed column S 440. In various embodiments, a first filter 540a (generally or collectively, filter 540) filters out particulates from the solvent stream between the first tank 510a and column S 440. In various embodiments, a fourth heat exchanger 530d and a seventh heat exchanger 530g are disposed between the first tank 510a and column S 440 to pre-heat the input stream for delivery to column S 440. For example, the fourth heat exchanger 530d may use the retentate stream as a hot stream against the cooler input stream, serving the dual purpose of pre-heating the input stream and cooling/condensing the retentate stream. In another example, the seventh heat exchanger 530g may use a bottom stream from column S as a hot stream against the cooler input stream, serving the dual purpose of pre-heating the input stream and cooling/condensing the bottoms stream before being collected for recycling or disposal from the plant.

In various embodiments, the bottoms stream of column S 440 is sent to an eighth heat exchanger 530h (e.g., a reboiler) that receives a hot stream to heat the bottoms stream. The eighth heat exchanger 530h returns at least a portion of the heated bottoms steam directly to column S 440 to drive distillation. The eighth heat exchanger 530h, via a second pump 520b, also directs a portion of the bottoms stream to a bottoms collection tank for recycling or disposal, and potentially for use as a hot stream (e.g., via the seventh heat exchanger 530g). The hot stream used to heat the bottoms stream in the eighth heat exchanger 530h may be directed to a third tank 510c.

The overhead stream from column S may be further heated via a first heat exchanger 530a against a hot stream and filtered by a second filter 540b before delivery to the membrane filter 450. The hot stream from the first heat exchanger 530a may be directed to a third tank 510c after exchanging heat with the input stream to the membrane filter 450. In various embodiments, the third tank 510c may be a condensate collection tank, which may allow a portion of the hot stream (e.g., from the first and eighth heat exchangers 530a/30h) that remains vaporous to be directed to the evaporators and a portion of the hot stream that has condensed to a liquid to be returned to the boilers via a fifth pump 520c.

The permeate output from the membrane filter 450 may be cooled against a cold stream in a second heat exchanger 530b before storage in a second tank 510b. The second tank 510b may be vented to the external environment via a third pump 520c, or recycled as an input for column S to supplement the wine from column A 410 by a fourth pump 520d moving the permeate to the first tank 510a to mix with the wine. Although illustrated in FIG. 5 as exchanging heat against a generic cold stream, the second heat exchanger 530b may be disposed in the piping pathway between column A 410a and column S 440 (e.g., with or instead of the fourth heat exchanger 530d and the seventh heat exchanger 530g) to use the input stream to column S 440 as a cold stream or be sent as a vapor to another distillation column 410 (e.g., as a driving vapor).

The retentate output from membrane filter 450 may be cooled against a cold stream in a third heat exchanger 530c or a fifth heat exchanger 530e before storage or provision as an output from the plant. In various embodiments, the third heat exchanger 530c condenses the vaporous retentate stream into a liquid, which is used as a hot steam in the fourth heat exchanger 530d to recover heat from the dehydration process by pre-heating the input stream to column S and further cooling the anhydrous solvent.

The described filter-based design provides a net consumption of steam of 0.4 kg of equivalent steam per liter of anhydrous ethanol produced, which represents a steam consumption reduction of ˜69% when compared to steam consumption in a typical cyclohexane-based design (from 1.3 to 0.4 kg of equivalent steam/L anhydrous). Additionally, the ability of the membrane filters to dehydrate streams at lower ethanol concentrations enables a reduction in the distillation section by omitting or bypassing a column B 410b. Therefore, an additional 0.8 kg of equivalent steam/L anhydrous of savings can be added to by the filter-based design, leading to a total steam consumption in distillation of 1.2 kg of equivalent steam/L anhydrous. As significantly less energy is needed in distillation and dehydration, there is less driving vapor demand across the plant and the pre-evaporator system 130 accordingly consumes less steam from the bagasse boilers. Both savings combined translate to a reduction in bagasse consumption of ˜ 45-50%, which can enable the plant to either produce more power for output the grid or the provision of bagasse as a product in the spot market.

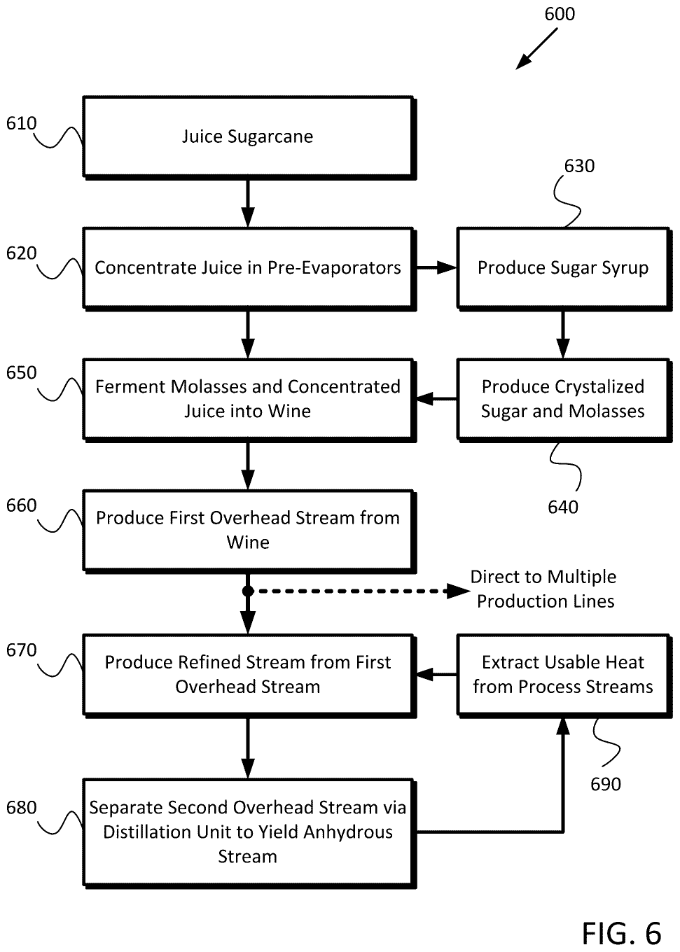

FIG. 6 is a flowchart of an example method 600 for operating a dual-process production plant 100, according to embodiments of the present disclosure.

At block 610, the system juices sugarcane, or another sugar-bearding feedstock, to produce a juice. In various embodiments, the system may slice, crush, masticate, or generally break the feedstock into smaller segments or pieces to release water and dissolved sugars from the feedstock. In various embodiments, the fluid level of the juice may be augmented by various digestive liquids (e.g., to further release water and dissolved sugars from the feedstock) or water (e.g., to ensure a fluid level in the juice supply system, to draw undissolved sugars from the feedstock, etc.). The juice therefore provides a mixture of sugar, water, and solids (e.g., portions of the feedstock).

At block 620, the system concentrates the juice (produced per block 610) in one or more evaporators of a pre-evaporator system. In various embodiments, the evaporators yield an evaporated juice having a higher concentration of sugar than the juice received from juice supply system (e.g., due to having a lower concentration of water than the juice—the water being driven off via evaporation powered by a steam).

At block 630, the system produces a sugar syrup in a sugar production sub-system from the concentrated juice (produced per block 620). The sugar syrup includes a higher concentration of sugar than the concentrated juice, due to the sugar production sub-system driving off water from the concentrated juice, using heat from a driving vapor received from the evaporators of the pre-evaporator system.

At block 640, the system produces crystallized sugar and molasses from the sugar syrup (produced per block 630). In various embodiments, various cooking vessels and centrifuges may be used to separate the sugar and molasses from one another and remaining water in the syrup. The separated sugar may be further dried, mixed with molasses in known ratios, formed into blocks/cones, etc. to produce a desired grade and form factor for the sugar.

At block 650, the system ferments one or both of molasses (produced per block 640) and the concentrated juice (per block 620) to produce a wine including an organic solvent (e.g., ethanol) from the sugar in the molasses and/or concentrated juice. In various embodiments, the wine includes various concentrations of the organic solvent, water, residual sugar, and various undesired off-products from the fermentation process (e.g., methanol, esters, spent yeast, etc.). A yeast or bacterial culture may be added to or maintained in the vessels in which fermentation takes place to consume the sugar in the juice and produce the desired solvent as a metabolic byproduct (e.g., ethanol). In an ethanol fermentation process, for example, the fermentation process produces a wine with 6-8 wt % of the desired solvent (e.g., 12 P-16 P ethanol).

At block 660, the system produces, in a distillation section, a first overhead stream from the wine (produced per block 650) having a higher concentration of the solvent than the wine. A wine column (distillation column A) receives the wine from the fermentation section, and using heat from driving vapors from a pre-evaporator section, distills a first overhead stream from the wine-leaving behind a vinasse, which may be extracted from the wine column as a bottoms stream. Unlike beer columns that operate below atmospheric pressures (e.g., at negative or vacuum pressures), the wine column operates at near-atmospheric pressures (e.g., about 1 barA) to produce an overhead stream. In various embodiments, the overhead stream has a concentration of solvent of 50, 50-55, 55, 55-60, 60, or 50-60 wt % (e.g., 100 P-120 P for ethanol).

In various embodiments, the distillation section includes a rectifier column (distillation column B), which further refines the solvent from the first overhead steam. Similarly to the wine column, the rectifier column uses heat from driving vapors from a pre-evaporator section, and produces a second overhead stream with a higher concentration of the solvent than the input stream. The rectifier column outputs flegmassa as a bottoms stream, and operates at near-atmospheric pressures (e.g., about 1 barA).

The piping of the system may be arranged and selectively configured to route the first overhead stream to multiple production lines, which may each use multiple instances of the same separation technology or instances of different separation technologies. For example, a first production line for an alcohol separation section may use a cyclohexane-based dehydration unit, a second production line for an alcohol separation section may also use a cyclohexane-based dehydration unit, and a third production line for an alcohol separation section may use a MEG-based, an MSU-based, or a filter-based dehydration unit. These different separation sections may receive inputs from individually corresponding distillation sections (e.g., N distillation sections for N separation sections) or may receive inputs from shared distillation sections (e.g., one distillation section serving 2-N separation sections).

At block 670, the system produces, in a solvent separation section, a refined stream from the first overhead stream and (optionally) one or more reflux streams from a dehydration unit.

When using a filter-based dehydration unit, a splitter column (distillation column S) receives the first overhead stream from the wine column, and produces a second overhead stream that the system directs to a membrane filter. The second overhead stream includes a lower concertation of water (and therefore a higher concentration of the solvent) than the first overhead stream (e.g., 100 P ethanol received versus 120 P ethanol output).

When using a cyclohexane-based dehydration unit, a separation column (distillation column C) receives the input steam from a rectifier column (distillation column B) in the distillation section, at a first concentration of the solvent at or near the azeotropic limit of water and that solvent (e.g., 190 P for ethanol and water) and an entrained overhead stream that includes an entrainer, such as cyclohexane.

When using a MEG-based dehydration unit, a separation column (distillation column C) receives the input steam from a rectifier column (distillation column B) in the distillation section, at a first concentration of the solvent at or near the azeotropic limit of water and that solvent (e.g., 190 P for ethanol and water) and an injection stream of MEG.

When using an MSU-based dehydration unit, an MSU receives the input steam from a rectifier column (distillation column B) in the distillation section, at a first concentration of the solvent at or near the azeotropic limit of water and that solvent (e.g., 190 P for ethanol and water) and a reflux stream of hydrous solvent from a separation column (distillation column C). The MSU directs one or more of a regen and a depressure stream to the separation column, which separates the hydrous solvent stream from the regen or depressure streams as an overhead stream to have a higher concentration of the solvent than the input stream(s). The separation column returns the hydrous solvent stream for additional processing to the MSU or provides the hydrous solvent stream as an intermediate product from the plant or one or more other systems in the plant. The separation column releases the water separated from the regen or depressure streams as a bottoms stream, with may be recycled or disposed of by the plant.

At block 680, the system separates the refined stream via a distillation unit to yield an anhydrous stream of the solvent having a higher concentration of the solvent than the refined stream.

When using a filter-based dehydration unit, the membrane filter receives a second overhead stream as a refined stream from the stripper column. The membrane filter separates, via active attraction differences to the different components of the second overhead stream, the second overhead stream into a permeate stream of undesired products (e.g., water) and a retentate stream of an anhydrous solvent (e.g., 200 P ethanol). In various embodiments, the permeate stream, which includes a higher concentration of water than the second overhead stream, may be returned as a reflux stream to the splitter column (e.g., as makeup fluid or to recapture heat), or may be discarded. In various embodiments, the retentate stream, which includes a higher concentration of a desired solvent than the refined stream (e.g., 120 P ethanol to 200 P ethanol) may be condensed and cooled for use as an output from the plant, or for use elsewhere in the plant (e.g., to refresh depleted MSU beds).

When using a cyclohexane-based dehydration unit, the separation column breaks apart the water-solvent azeotrope by creating a new azcotrope between the entrainer (e.g., cyclohexane) and water. The separation column removes this new azeotrope as an overhead stream of the entrained water to leave behind the anhydrous solvent (e.g., 200 P ethanol), which is output as a bottom stream from the separation column and an overhead stream of the newly formed azeotrope (e.g., water, cyclohexane, and residual ethanol).

The entrained stream of the new azeotrope may be directed to a recovery column (distillation column D) to separate the cyclohexane from the azeotropic mixture, which may then be returned (optionally with makeup fresh cyclohexane) as a reflux stream to the separation column to continue the separation process.

When using a MEG-based dehydration unit, the separation column breaks apart the water-solvent azeotrope by creating a new azeotrope between the MEG and water. The separation column removes this new azeotrope as an entrained bottoms stream of the MEG and water to leave behind the anhydrous solvent (e.g., 200 P ethanol), which is output as an overhead stream from the separation column.

The entrained stream of the new azeotrope may be directed to a recovery column (distillation column D) to separate the MEG from the azeotropic mixture, which may then be returned as a reflux stream to the separation column to continue the separation process.

When using an MSU-based dehydration unit, the MSU passes the input stream through one or more beds that separate the desired solvent from the input stream to output a stream of the anhydrous solvent.

At block 690, the system extracts usable heat from the process streams. For example the anhydrous solvent stream is generally stored as a liquid, but is output as a vapor from various dehydration units. Similarly, the various distillation columns generally heat the respective input streams to produce an overhead vapor output. Accordingly, a hotter anhydrous solvent stream can be both cooled/condensed for storage and the input streams to various distillation columns can be pre-heated to reduce the need for external cold streams or external hot streams (and for heating in the various distillation columns) by exchanging heat from the (hotter) output process streams from the dehydration units against the (colder) input streams for the separation section or other sections in the dual-process production plant.

For example, when using a filter-based dehydration unit, one or more of the bottoms stream from the distillation column in the separation system, the permeate stream from the membrane filter, or the retentate stream from the membrane filter may be exchange heat against the input stream to the distillation column to pre-heat the input stream and cool the process streams; thereby conserving energy in the plant.

The present disclosure may also be understood with reference to the following numbered clauses:

Clause 1: A production plant, comprising: a juice supply; a first evaporator system; a sugar production system, comprising: a second evaporator system; and a sugar separation section; an alcohol production section, comprising: a fermentation section; a distillation section; and an alcohol separation section, including a distillation column and a dehydration unit; and piping, arranged to selectively configure: the first evaporator system to receive juice from the juice supply, the juice providing a mixture of sugar, water, and solids; the second evaporator system to receive a first portion of an evaporated juice having a higher concentration of sugar than the juice from the first evaporator system; the sugar separation section to receive syrup from the second evaporator system having a higher concentration of sugar than the evaporated juice; the fermentation section to receive molasses from the sugar separation section and a second portion of the evaporated juice from the first evaporator system including a higher concentration of sugar than the juice; the distillation section to receive, from the fermentation section, a wine including an alcohol produced from the molasses and the evaporated juice; the distillation column to receive, from the distillation section, a first overhead stream having a higher concentration of alcohol than the wine; and the dehydration unit to receive, from the distillation column, a second overhead stream having a higher concentration of alcohol than the first overhead stream and to output an anhydrous stream having a higher concentration of alcohol than the second overhead stream.

Clause 2: The production plant as described in any of clauses 1 and 3-11, further comprising: a dryer; and wherein the piping is further arranged to selectively configure the dryer to receive a wet sugar from the sugar separation section and output a sugar stream having a lower concentration of water than the wet sugar.

Clause 3: The production plant as described in any of clauses 1-2 and 4-11, further comprising vapor ducting, arranged to selectively configure: the second evaporator system to receive a first portion of a driving vapor produced by the first evaporator system; and the distillation section to receive a second portion of the driving vapor.

Clause 4: The production plant as described in any of clauses 1-3 and 5-11, further comprising a boiler, wherein the piping is configured to: direct steam from the boiler to the distillation column to drive generation of the second overhead stream; direct condensate of the steam directed to the distillation column back to the boiler; and direct bagasse produced by the juice supply to a fuel input for the boiler.

Clause 5: The production plant as described in any of clauses 1-4 and 6-11, wherein the dehydration unit includes a membrane filter configured to receive an input stream and output a permeate stream having a higher concentration of water than the first overhead stream; wherein the piping is further arranged to selectively configure the distillation column to receive the permeate stream.

Clause 6: The production plant as described in any of clauses 1-5 and 7-11, wherein: the distillation section includes: a wine column; and a rectifier column; the alcohol production section further includes: a separation column; and a recovery column; and the piping is further arranged to selectively configure: the wine column to receive the wine from the fermentation section; the rectifier column to receive a concentrated wine vapor from the wine column, having a higher concentration of alcohol than the wine; the separation column to receive a hydrous solvent stream from the rectifier column, having a higher concentration of alcohol than the concentrated wine vapor, to receive an entrainer stream including cyclohexane from the recovery column, and to output the anhydrous stream as a bottoms stream from the separation column; and the recovery column to receive an entrained overhead stream from the separation column including water and cyclohexane.

Clause 7: The production plant as described in any of clauses 1-6 and 8-11, wherein: the distillation section includes: a wine column; and a rectifier column; the alcohol production section further includes: a separation column; and a recovery column; and the piping is further arranged to selectively configure: the wine column to receive the wine from the fermentation section; the rectifier column to receive a concentrated wine vapor from the wine column, having a higher concentration of alcohol than the wine; the separation column to receive a hydrous solvent stream from the rectifier column, having a higher concentration of alcohol than the concentrated wine vapor, to receive an injection stream including monoethyleneglycol (MEG) from the recovery column, and to output the anhydrous stream as an overhead stream from the separation column; and the recovery column to receive an entrained bottoms stream from the separation column including water and cyclohexane.

Clause 8: The production plant as described in any of clauses 1-7 and 9-11, wherein the dehydration unit includes a molecular sieve unit (MSU) configured to receive an input stream and output a product stream of a lower water concentration than the input stream; and wherein the piping is further arranged to selectively configure the MSU to receive the input stream of one or both of the first overhead stream and the second overhead stream.

Clause 9: The production plant as described in any of clauses 1-8 and 10-11, further comprising a heat exchanger disposed between the distillation section and the distillation column, the piping further arranged to configure the first overhead stream to provide thermal energy to a cold stream passing though the heat exchanger.

Clause 10: The production plant as described in any of clauses 1-9 and 11, wherein the distillation section operates between 1-2 bar of pressure to distill the first overhead stream from the wine.

Clause 11: The production plant as described in any of clauses 1-10, wherein the dehydration unit receives the second overhead stream at between 40-70% weight alcohol.

Clause 12: An alcohol production system, comprising: an evaporator system; a fermentation section; a distillation section; a distillation column; a dehydration unit; and piping, arranged to selectively configure: the evaporator system to receive a juice stream providing a mixture of sugar, water, and solids; the fermentation section to receive, from the evaporator system, a concentrated juice stream having a lower concentration of water than the juice stream; the distillation section to receive, from the fermentation section, a wine providing a mixture of sugar, water, solids, and alcohol; the distillation column to receive, from the distillation section, a first overhead stream produced by the distillation section having a lower concentration of sugars and solids and a lower concentration of water than the wine; and the dehydration unit to receive, from the distillation column, a second overhead stream produced by the distillation column having a lower concentration of water than the first overhead stream and to output a retentate stream having a higher concentration of alcohol than the second overhead stream.

Clause 13: The alcohol production system as described in any of clauses 12 and 14-18, wherein the concentrated juice stream received by the fermentation section is a first portion of the concentrated juice stream, and the piping is further configured to direct a second portion of the concentrated juice stream to a sugar separation section.

Clause 14: The alcohol production system as described in any of clauses 12-13 and 15-18, further comprising: a rectifier column; a separation column; and a recovery column; wherein the first overhead stream received by the distillation column is a first portion of the first overhead stream, and the piping is further arranged to selectively configure: the rectifier column to receive a second portion of the first overhead stream; the separation column to: receive, from the rectifier column, a hydrous alcohol stream having a lower concentration of water than the first overhead stream; receive, from the recovery column, a cyclohexane stream; and output, an anhydrous ethanol bottoms stream having a higher concentration of alcohol than the hydrous alcohol stream; and the recovery column to receive, from the separation column, a recycled overhead stream including cyclohexane and having a higher concentration of water than the hydrous alcohol stream.

Clause 15: The alcohol production system as described in any of clauses 12-14 and 16-18, further comprising: a rectifier column; a separation column; and a recovery column; wherein the first overhead stream received by the distillation column is a first portion of the first overhead stream, and the piping is further arranged to selectively configure: the rectifier column to receive a second portion of the first overhead stream; the separation column to: receive, from the rectifier column, a hydrous alcohol stream having a lower concentration of water than the first overhead stream; receive, from the recovery column, a monocthyleneglycol (MEG) stream; and output, an anhydrous ethanol overhead stream having a higher concentration of alcohol than the hydrous alcohol stream; and the recovery column to receive, from the separation column, a recycled bottoms stream including MEG and having a higher concentration of water than the hydrous alcohol stream.

Clause 16: The alcohol production system as described in any of clauses 12-15 and 17-18, further comprising a heat exchanger, the piping further arranged to configure the retentate stream to transfer thermal energy as a hot stream passing through the heat exchanger to another stream in the production system having a lower initial temperature when entering the heat exchanger than the retentate stream when entering the heat exchanger.

Clause 17: The alcohol production system as described in any of clauses 12-16 and 18, wherein the distillation section includes a wine column that operates at or above atmospheric pressure to produce the first overhead stream.

Clause 18: The alcohol production system as described in any of clauses 12-17, wherein the first overhead stream consists of 120 proof alcohol.

Clause 19: A dual-process solvent and solids production system, comprising: a first evaporator system, configured to receive a feed stream including a dissolved solid and water; a solids production system, comprising: a second evaporator system; and a first separation system; a solvent production system, comprising: a fermentation section; a distillation section; and a solvent separation section, including a distillation column, a dehydration unit; and piping, arranged to selectively configure: the second evaporator system to receive, from the first evaporator system, a first portion of an evaporated feed stream having a higher concentration of the dissolved solid than the feed stream; the solids production system to receive syrup from the second evaporator system having a higher concentration of the dissolved solid than the evaporated feed stream; the fermentation section to receive, from the first evaporator system, a second portion of the evaporated feed stream; the distillation section to receive, from the fermentation section, a solvent stream including a solvent produced from the evaporated feed stream; the distillation column to receive, from the distillation section, a first overhead stream having a higher concentration of the solvent than the solvent stream and to receive, from the dehydration unit, a permeate stream having a higher concentration of water than the first overhead stream; and the distillation section to receive, from the distillation column, a second overhead stream having a higher concentration of the solvent than the first overhead stream and to output a retentate stream having a higher concentration of the solvent than the second overhead stream.

Clause 20: The dual-process solvent and solids production system as described in any of clauses 19 and 21, wherein the distillation section operates between 1-2 bar of pressure to distill the first overhead stream from the solvent stream.

Clause 21: The dual-process solvent and solids production system as described in any of clauses 19 and 20, wherein the first overhead stream consists of 120 proof alcohol.

Clause 22: A method, comprising: receiving, at a first evaporator system, a juice providing a mixture of sugar, water, and solids; producing, at the first evaporator system, an evaporated juice having a higher concentration of sugar than the juice; receiving, at a second evaporator system from the first evaporator system, a first portion of the evaporated juice; producing, at the second evaporator system, a syrup having a higher concentration of sugar than the evaporated juice; receiving, at a sugar separation section from the second evaporator system, the syrup; producing, at the sugar separation section, a crystalized sugar and a molasses; receiving, at a fermentation section, the molasses and a second portion of the evaporated juice; fermenting, at the fermentation section the molasses and the evaporated juice to produce a wine including an organic solvent; receiving, at a distillation section from the fermentation section, the wine; heating, at the distillation section operating between 1-2 bar of pressure, the wine to produce a first overhead stream having a higher concentration of the organic solvent than the wine; receiving, at a distillation column from the distillation section, the first overhead stream; separating, at the distillation column, a second overhead stream from the first overhead stream that has a higher concentration of the solvent than the first overhead stream and that is less than or equal to 60% by weight of the solvent; receiving, at a dehydration unit from the distillation column, the second overhead stream; and separating, at the dehydration unit, the second overhead stream into a retentate stream having a higher concentration of the solvent and the second overhead stream and into a permeate stream, having a lower concentration of the solvent than the second overhead stream.

Unless otherwise indicated, all numbers expressing quantities of ingredients, properties such as molecular weight, reaction conditions, and so forth used in the specification and claims are to be understood as being modified in all instances by the term “about.” Accordingly, unless indicated to the contrary, the numerical parameters set forth in the following specification and attached claims are approximations that may vary depending upon the desired properties sought to be obtained by the present invention. At the very least, and not as an attempt to limit the application of the doctrine of equivalents to the scope of the claims, each numerical parameter should at least be construed in light of the number of reported significant digits and by applying ordinary rounding techniques. Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in their respective testing measurements.

As used herein, “about,” “approximately” and “substantially” are understood to refer to numbers in a range of the referenced number, for example the range of-10% to +10% of the referenced number, preferably-5% to +5% of the referenced number, more preferably-1% to +1% of the referenced number, most preferably-0.1% to +0.1% of the referenced number.

Furthermore, all numerical ranges herein should be understood to include all integers, whole numbers, or fractions, within the range. Moreover, these numerical ranges should be construed as providing support for a claim directed to any number or subset of numbers in that range. For example, a disclosure of “from 1 to 10” should be construed as also supporting a range of from 1 to 8, from 3 to 7, from 1 to 9, from 3.6 to 4.6, from 3.5 to 9.9, and so forth.

The terms “a” and “an” and “the” and similar referents used in the context of describing the invention (especially in the context of the following claims) are to be construed to cover both the singular and the plural, unless otherwise indicated herein or clearly contradicted by context. Recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g. “such as”) provided herein is intended merely to better illuminate the invention and does not pose a limitation on the scope of the invention otherwise claimed. No language in the specification should be construed as indicating any non-claimed element essential to the practice of the invention.

The use of the term “or” in the claims is used to mean “and/or” unless explicitly indicated to refer to alternatives only or the alternatives are mutually exclusive, although the disclosure supports a definition that refers to only alternatives and “and/or.”

Groupings of alternative elements or aspects of the invention disclosed herein are not to be construed as limitations. Each group member may be referred to and claimed individually or in any combination with other members of the group or other elements found herein. It is anticipated that one or more members of a group may be included in, or deleted from, a group for reasons of convenience and/or patentability. When any such inclusion or deletion occurs, the present disclosure is herein deemed to contain the group as modified thus fulfilling the written description of all Markush groups used in the appended claims.

Preferred embodiments of this invention are described herein, including the best mode known to the inventors for carrying out the invention. Of course, variations on those preferred embodiments will become apparent to those of ordinary skill in the art upon reading the foregoing description. The inventors expect those of ordinary skill in the art to employ such variations as appropriate, and the inventors intend for the inventive concepts of the present disclosure to be practiced otherwise than specifically described herein. Accordingly, the present disclosure includes all modifications and equivalents of the subject matter recited in the claims appended hereto as permitted by applicable law. Moreover, any combination of the above-described elements in all possible variations thereof is encompassed by the present disclosure unless otherwise indicated herein or otherwise clearly contradicted by context.

Specific aspects disclosed herein may be further limited in the claims using consisting of or consisting essentially of language. When used in the claims, whether as filed or added per amendment, the transition term “consisting of” excludes any element, step, or ingredient not specified in the claims. The transition term “consisting essentially of” limits the scope of a claim to the specified materials or steps and those that do not materially affect the basic and novel characteristic(s). Aspects of the invention so claimed are inherently or expressly described and enabled herein.

As used in the present disclosure, a phrase referring to “at least one of” a list of items refers to any set of those items, including sets with a single member, and every potential combination thereof. For example, when referencing “at least one of A, B, or C” or “at least one of A, B, and C”, the phrase is intended to cover the sets of: A, B, C, A-B, B-C, and A-B-C, where the sets may include one or multiple instances of a given member (e.g., A-A, A-A-A, A-A-B, A-A-B-B-C-C-C, etc.) and any ordering thereof. For avoidance of doubt, the phrase “at least one of A, B, and C” shall not be interpreted to mean “at least one of A, at least one of B, and at least one of C”.

Within the claims, reference to an element in the singular is not intended to mean “one and only one” unless specifically stated as such, but rather as “one or more” or “at least one”. Unless specifically stated otherwise, the term “some” refers to one or more. No claim element is to be construed under the provision of 35 U.S.C. § 112(f) unless the element is expressly recited using the phrase “means for” or “step for”. All structural and functional equivalents to the elements of the various embodiments described in the present disclosure that are known or come later to be known to those of ordinary skill in the relevant art are expressly incorporated herein by reference and are intended to be encompassed by the claims. Moreover, nothing disclosed in the present disclosure is intended to be dedicated to the public regardless of whether such disclosure is explicitly recited in the claims.

Further, it is believed that one skilled in the art can use the preceding description to use the claimed inventions to their fullest extent. The examples and aspects disclosed herein are to be construed as merely illustrative and not a limitation of the scope of the present disclosure in any way. It will be apparent to those having skill in the art that changes may be made to the details of the above-described examples without departing from the underlying principles discussed. In other words, various modifications and improvements of the examples specifically disclosed in the description above are within the scope of the appended claims. For instance, any suitable combination of features of the various examples described is contemplated.

The invention is claimed as follows:

1. A production plant, comprising:
a juice supply;
a first evaporator system;
a sugar production system, comprising:
a second evaporator system; and
a sugar separation section;
an alcohol production section, comprising:
a fermentation section;
a distillation section; and
an alcohol separation section, including a distillation column and a dehydration unit; and
piping, arranged to selectively configure:
the first evaporator system to receive juice from the juice supply, the juice providing a mixture of sugar, water, and solids;
the second evaporator system to receive a first portion of an evaporated juice having a higher concentration of sugar than the juice from the first evaporator system;
the sugar separation section to receive syrup from the second evaporator system having a higher concentration of sugar than the evaporated juice;
the fermentation section to receive molasses from the sugar separation section and a second portion of the evaporated juice from the first evaporator system including a higher concentration of sugar than the juice;
the distillation section to receive, from the fermentation section, a wine including an alcohol produced from the molasses and the evaporated juice;
the distillation column to receive, from the distillation section, a first overhead stream having a higher concentration of alcohol than the wine; and
the dehydration unit to receive, from the distillation column, a second overhead stream having a higher concentration of alcohol than the first overhead stream and to output an anhydrous stream having a higher concentration of alcohol than the second overhead stream.

2. The production plant of claim 1, further comprising:
a dryer; and
wherein the piping is further arranged to selectively configure the dryer to receive a wet sugar from the sugar separation section and output a sugar stream having a lower concentration of water than the wet sugar.

3. The production plant of claim 1, further comprising vapor ducting, arranged to selectively configure:
the second evaporator system to receive a first portion of a driving vapor produced by the first evaporator system; and
the distillation section to receive a second portion of the driving vapor.

4. The production plant of claim 1, further comprising a boiler, wherein the piping is configured to:
direct steam from the boiler to the distillation column to drive generation of the second overhead stream;
direct condensate of the steam directed to the distillation column back to the boiler; and
direct bagasse produced by the juice supply to a fuel input for the boiler.

5. The production plant of claim 1, wherein the dehydration unit includes a membrane filter configured to receive an input stream and output a permeate stream having a higher concentration of water than the first overhead stream;
wherein the piping is further arranged to selectively configure the distillation column to receive the permeate stream.

6. The production plant of claim 1, wherein:
the distillation section includes:
a wine column; and
a rectifier column;
the alcohol production section further includes:
a separation column; and
a recovery column; and
the piping is further arranged to selectively configure:
the wine column to receive the wine from the fermentation section;
the rectifier column to receive a concentrated wine vapor from the wine column, having a higher concentration of alcohol than the wine;
the separation column to receive a hydrous solvent stream from the rectifier column, having a higher concentration of alcohol than the concentrated wine vapor, to receive an entrainer stream including cyclohexane from the recovery column, and to output the anhydrous stream as a bottoms stream from the separation column; and
the recovery column to receive an entrained overhead stream from the separation column including water and cyclohexane.

7. The production plant of claim 1, wherein:
the distillation section includes:
a wine column; and
a rectifier column;
the alcohol production section further includes:
a separation column; and
a recovery column; and
the piping is further arranged to selectively configure:
the wine column to receive the wine from the fermentation section;
the rectifier column to receive a concentrated wine vapor from the wine column, having a higher concentration of alcohol than the wine;
the separation column to receive a hydrous solvent stream from the rectifier column, having a higher concentration of alcohol than the concentrated wine vapor, to receive an injection stream including monoethyleneglycol (MEG) from the recovery column, and to output the anhydrous stream as an overhead stream from the separation column; and
the recovery column to receive an entrained bottoms stream from the separation column including water and cyclohexane.

8. The production plant of claim 1, wherein the dehydration unit includes a molecular sieve unit (MSU) configured to receive an input stream and output a product stream of a lower water concentration than the input stream; and
wherein the piping is further arranged to selectively configure the MSU to receive the input stream of one or both of the first overhead stream and the second overhead stream.

9. The production plant of claim 1, further comprising a heat exchanger disposed between the distillation section and the distillation column, the piping further arranged to configure the first overhead stream to provide thermal energy to a cold stream passing though the heat exchanger.

10. The production plant of claim 1, wherein the distillation section operates between 1-2 bar of pressure to distill the first overhead stream from the wine.

11. The production plant of claim 1, wherein the dehydration unit receives the second overhead stream at between 40-70% weight alcohol.

12. An alcohol production system, comprising:
an evaporator system;
a fermentation section;
a distillation section;
a distillation column;
a dehydration unit; and
piping, arranged to selectively configure:
the evaporator system to receive a juice stream providing a mixture of sugar, water, and solids;
the fermentation section to receive, from the evaporator system, a concentrated juice stream having a lower concentration of water than the juice stream;
the distillation section to receive, from the fermentation section, a wine providing a mixture of sugar, water, solids, and alcohol;
the distillation column to receive, from the distillation section, a first overhead stream produced by the distillation section having a lower concentration of sugars and solids and a lower concentration of water than the wine; and
the dehydration unit to receive, from the distillation column, a second overhead stream produced by the distillation column having a lower concentration of water than the first overhead stream and to output a retentate stream having a higher concentration of alcohol than the second overhead stream.

13. The alcohol production system of claim 12, wherein the concentrated juice stream received by the fermentation section is a first portion of the concentrated juice stream, and the piping is further configured to direct a second portion of the concentrated juice stream to a sugar separation section.

14. The alcohol production system of claim 12, further comprising:
a rectifier column;
a separation column; and
a recovery column;
wherein the first overhead stream received by the distillation column is a first portion of the first overhead stream, and the piping is further arranged to selectively configure:
the rectifier column to receive a second portion of the first overhead stream;
the separation column to:
receive, from the rectifier column, a hydrous alcohol stream having a lower concentration of water than the first overhead stream;
receive, from the recovery column, a cyclohexane stream; and
output, an anhydrous ethanol bottoms stream having a higher concentration of alcohol than the hydrous alcohol stream; and
the recovery column to receive, from the separation column, a recycled overhead stream including cyclohexane and having a higher concentration of water than the hydrous alcohol stream.

15. The alcohol production system of claim 12, further comprising:
a rectifier column;
a separation column; and
a recovery column;
wherein the first overhead stream received by the distillation column is a first portion of the first overhead stream, and the piping is further arranged to selectively configure:
the rectifier column to receive a second portion of the first overhead stream;
the separation column to:
receive, from the rectifier column, a hydrous alcohol stream having a lower concentration of water than the first overhead stream;
receive, from the recovery column, a monoethyleneglycol (MEG) stream; and
output, an anhydrous ethanol overhead stream having a higher concentration of alcohol than the hydrous alcohol stream; and
the recovery column to receive, from the separation column, a recycled bottoms stream including MEG and having a higher concentration of water than the hydrous alcohol stream.

16. The alcohol production system of claim 12, further comprising a heat exchanger, the piping further arranged to configure the retentate stream to transfer thermal energy as a hot stream passing through the heat exchanger to another stream in the alcohol production system having a lower initial temperature when entering the heat exchanger than the retentate stream when entering the heat exchanger.

17. The alcohol production system of claim 12, wherein the distillation section includes a wine column that operates at or above atmospheric pressure to produce the first overhead stream.

18. The alcohol production system of claim 12, wherein the first overhead stream consists of 120 proof alcohol.

19. A dual-process solvent and solids production system, comprising:
a first evaporator system, configured to receive a feed stream including a dissolved solid and water;
a solids production system, comprising:
a second evaporator system; and
a first separation system;
a solvent production system, comprising:
a fermentation section;
a distillation section; and
a solvent separation section, including a distillation column, a dehydration unit; and
piping, arranged to selectively configure:
the second evaporator system to receive, from the first evaporator system, a first portion of an evaporated feed stream having a higher concentration of the dissolved solid than the feed stream;
the solids production system to receive syrup from the second evaporator system having a higher concentration of the dissolved solid than the evaporated feed stream;
the fermentation section to receive, from the first evaporator system, a second portion of the evaporated feed stream;
the distillation section to receive, from the fermentation section, a solvent stream including a solvent produced from the evaporated feed stream;
the distillation column to receive, from the distillation section, a first overhead stream having a higher concentration of the solvent than the solvent stream and to receive, from the dehydration unit, a permeate stream having a higher concentration of water than the first overhead stream; and
the distillation section to receive, from the distillation column, a second overhead stream having a higher concentration of the solvent than the first overhead stream and to output a retentate stream having a higher concentration of the solvent than the second overhead stream.

20. The dual-process solvent and solids production system of claim 19, wherein the distillation section operates between 1-2 bar of pressure to distill the first overhead stream from the solvent stream.

21. The dual-process solvent and solids production system of claim 19, wherein the first overhead stream consists of 120 proof alcohol.

22. A method, comprising:
receiving, at a first evaporator system, a juice providing a mixture of sugar, water, and solids;
producing, at the first evaporator system, an evaporated juice having a higher concentration of sugar than the juice;
receiving, at a second evaporator system from the first evaporator system, a first portion of the evaporated juice;
producing, at the second evaporator system, a syrup having a higher concentration of sugar than the evaporated juice;
receiving, at a sugar separation section from the second evaporator system, the syrup;
producing, at the sugar separation section, a crystalized sugar and a molasses;
receiving, at a fermentation section, the molasses and a second portion of the evaporated juice;
fermenting, at the fermentation section the molasses and the evaporated juice to produce a wine including an organic solvent;
receiving, at a distillation section from the fermentation section, the wine;
heating, at the distillation section operating between 1-2 bar of pressure, the wine to produce a first overhead stream having a higher concentration of the organic solvent than the wine;
receiving, at a distillation column from the distillation section, the first overhead stream;
separating, at the distillation column, a second overhead stream from the first overhead stream that has a higher concentration of the organic solvent than the first overhead stream and that is less than or equal to 60% by weight of the organic solvent;
receiving, at a dehydration unit from the distillation column, the second overhead stream; and
separating, at the dehydration unit, the second overhead stream into a retentate stream having a higher concentration of the organic solvent and the second overhead stream and into a permeate stream, having a lower concentration of the organic solvent than the second overhead stream.
